(19) United States
(12) Patent Application Publication
(10) Pub. No.: US 2006/0293924 A1
(43) Pub. Date: Dec. 28, 2006

(54) System and method for recording and auditing medical information

(75) Inventor: Allan Gardiner, Kensington, CA (US)

Correspondence Address: FLIESLER MEYER, LLP, FOUR EMBARCADERO CENTER, SUITE 400, SAN FRANCISCO, CA, 94111, US

(73) Assignee: ALPHA GOLF, INC., KENSINGTON, CA (US)

(21) Appl. No.: 11/433,839

(22) Filed: May 12, 2006

Related U.S. Application Data
(60) Provisional application No. 60/680,946, filed on May 13, 2005.

(51) Int. Cl. A61B 5/00; G06F 17/30

(52) U.S. Cl. 705/3; 707/8

(57) ABSTRACT

Systems and methods are provided for audio and video auditing and recording of medical information, particularly in a medical or other setting, and when the information is of a subject or patient. In accordance with one embodiment, the invention comprises a system having a display, a recording interface and a database storage. The display is used to display an electronic version of a questionnaire to be given to or asked by an interviewer of the subject or patient (the interviewee). A recording interface can be either audio, video, or a combination of both. Additionally, information provided by one or more measurement devices can be recorded in a database, along with a time stamp to provide an audit trail for the medical information. The database storage saves a copy of the questions as a self-assembling database together with an audio and/or video copy of the answers given by the patient to each question. During an interview, as each question is posed to the patient, a copy of the patients answer is recorded. Systems and methods can include one or more questions and answers, and can include information obtained from one or more measurement devices.

### CLAIM OF PRIORITY

This application claims priority under 35 U.S.C. §119(e) to U.S. Provisional Patent Application No: 60/680,946, filed May 13, 2005. This application is expressly incorporated herein fully by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The invention is generally related to techniques for recording interviews and case-related information in a medical or other setting, and more particularly to a system and method for audio and video recording and auditing of patient medical case histories.

### BACKGROUND

In a medical setting, such as within a hospital, clinic, medical-device manufacturer, or pharmaceutical industry, clinical trials are often used to test new drug therapies or treatments. During a clinical trial, a group of patients are treated with the new therapy, while their initial symptoms, treatment, and the results of their treatment carefully monitored, usually through a series of tests and patient interviews. Often, different patients will receive different dosages or combinations of therapies, to determine the optimal combination. The results from the clinical trial are then used to create treatment plans that have general applicability in the population as a whole. A satisfactory result may also be a prerequisite for obtaining government (FDA) approval for any new drug or medical procedure. This means that the clinical trial plays an important part in the commercial success of any new therapy.

During a typical clinical trial, and especially during a high-risk clinical trial where there is a risk to the patients health, two or more people may be present during any patient interview, to ensure that the results of the trial are accurately recorded. A patient's answers during the interview are also recorded on multi-part forms that can be used for basic auditing purposes. However, it will be seen that this technique is only as reliable as the person recording the patients answers. If an answer is incorrectly recorded on the multi-part form by one or more of the interviews, then the entire clinical trial may be skewed. In addition, the process is time-consuming and requires that more people be present than necessary, to ensure patient confidence levels. Ultimately, clinical trials that use this technique will cost more money, and take longer to complete, while still lacking 100% accuracy as to the interview answers.

Problems described above with respect to a medical setting are also present in other settings. Commercial trials and tests, such as product market research, mechanical prototype, or other forms of manufacturing may also include a clinical trial-like interview process, where the results or feedback from customers are used to enhance the product in some way. These commercial trials may also suffer from the problem of not being able to definitively or accurately record a customers interview answers during the trial.

### SUMMARY

The invention is generally related to techniques for recording interviews and case-related information in a medical or other setting, and more particularly to a system and method for audio and video recording and auditing of patient information. In accordance with one embodiment, the invention comprises a system having a display, a recording interface and a database storage. The display is used to display an electronic version of the questionnaire to be self-completed or asked by an interviewer of the patient (the interviewee). The recording interface can be either audio, video, or a combination of both. The database storage saves a copy of the questionnaire together with an audio and/or video copy of the answers given by the patient to each question. The entire system may be provided as a portable or portable desktop application (PDA) device. During an interview, as each question is posed to the patient, a copy of the patients answer is recorded. Voice recognition can permit the subject's answer to be compared with the entered data. If the questionnaire includes an electronic image, then depending on the file format used, the patients answer can be recorded with, or embedded into, that image. The system also includes a clock and a time stamp feature that stamps the answers with a time stamp. During any subsequent auditing of a patients results, the time stamp feature can be used by the auditor to quickly locate the backing recording given by the patient to any particular answer. The result is a system that allows for quick interviewing of a patient, by a single interviewer, yet ensures that a patients answers are reliably recorded in real-time, and with the confidence that a backing recording is kept for auditing purposes.

### BRIEF DESCRIPTION OF THE FIGURES

FIG. 1 shows an illustration of a system for audio and video recording and auditing of case histories and interviews in accordance with an embodiment of the present invention.

FIG. 2 shows a flowchart of a method for audio and video recording and auditing of case histories and interviews, in accordance with an embodiment of the present invention.

FIG. 3 shows an illustration of a system for audio and video recording and auditing of case histories and interviews, in accordance with an alternate embodiment of the present invention.

FIG. 4 illustrates an example of how a system may be used in a medical or other interview setting, in accordance with an embodiment of the present invention.

FIG. 5 shows a flowchart of a method of capturing audio video during a multiple question interview, together with voice recognition, in accordance with an embodiment of the present invention.

FIG. 6 shows a flowchart of a method of recording and displaying gait change progress of an interviewee, in accordance with an embodiment of the present invention.

FIG. 7 shows a flowchart of a method of using voice recognition to audit answers, in accordance with an embodiment of the present invention.

FIG. 8 shows a flowchart of a method of conducting a review operation, in accordance with an embodiment of the present invention.

FIG. 9 shows a flowchart of a method of conducting an audit operation, in accordance with an embodiment of the present invention.

FIG. 10 shows a flowchart of a method of training the voice recognition system, in accordance with an embodiment of the present invention.

FIG. 11 shows a flowchart of a method of allowing an interviewee to conduct a self-interview or self-questionnaire, in accordance with an embodiment of the present invention.

### DETAILED DESCRIPTION

The invention is generally related to techniques for recording interviews and case-related information in a medical or other setting, and more particularly to a system and method for audio and video recording and auditing of patient medical case histories. In accordance with one embodiment, the invention comprises a system having a display, a recording interface and a database storage. The display is used to display an electronic version of the questionnaire to be given to or asked by an interviewer of the patient (the interviewee). The recording interface can be either audio, video, or a combination of both. The database storage saves a copy of the questionnaire together with an audio and/or video copy of the answers given by the patient to each question. The entire system may be provided as a portable or portable desktop application (PDA) device. During an interview, as each question is posed to the patient, a copy of the patients answer is recorded. If the questionnaire includes an electronic image, then depending on the file format used the patients answer can be recorded with, or embedded into, that image. The system also includes a clock and a time stamp feature that stamps the answers and optionally, the audio with a time stamp. During any subsequent auditing of a patients results, the time stamp feature can be used by the auditor to quickly locate the backing recording given by the patient to any particular answer. The result is a system that allows for quick interviewing of a patient, by a single interview, yet ensures that a patients answers are reliably recorded in real-time, and with the confidence that a backing recording is kept for auditing purposes.

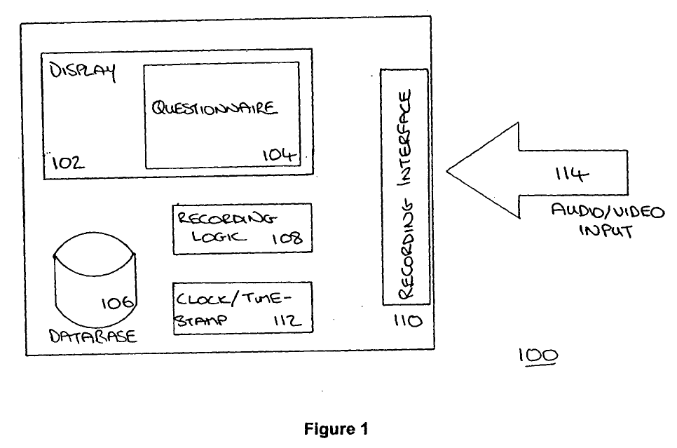

FIG. 1 shows an illustration of a system for audio and video recording and auditing of case histories and interviews in accordance with an embodiment of the present invention. As shown in FIG. 1, in accordance with an embodiment, the system 100 comprises a display 102, such as a video display, LCD display, or other form of display, for displaying an electronic version of the questionnaire 104. The questionnaire includes the questions that are to be asked of the subject (the interviewee). The questions may be verbally asked by the interviewer for the audio record, read by computer generated voice read by prerecorded voice, to prompt the interviewee to say and mark their own responses or way their response for the interviewer to mark on the electronic questionnaire. The system also comprises a recording interface 110. As the interviewee answers questions, their answers are captured as audio and/or video input 114, and communicated to the system via the recording interface. A recording logic 108 controls the entire process. The interviewees answers, and any supporting data, such as image files and/or data from the questionnaire, are stored within the system in a database storage 106. In accordance with an embodiment, a clock and time stamping feature 112 is provided to timestamp the answers, for assistance in any subsequent auditing, and as further described below.

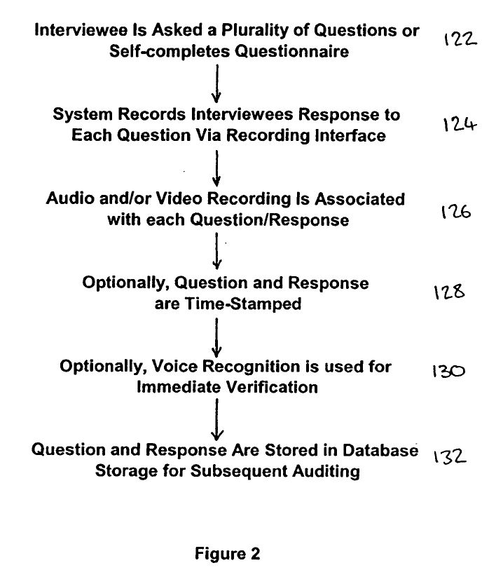

FIG. 2 shows a flowchart of a method for audio and video recording and auditing of case histories and interviews, in accordance with an embodiment of the present invention. As shown in FIG. 2, in step 122, the interviewee is either asked questions by the interviewer, or self-completes a questionnaire displayed on the systems display. In step 124, the system records the interviewees responses in either an audio and/or video input via the recording interface. In step 126, the question and response (together with the audio/video recording) are time-stamped, for subsequent use in playback or auditing In step 128, the audio and/or video recording is associated with the question/response pair. In step 130, voice recognition is optionally used for immediate verification, to compare the interviewees verbal answer with the answer recorded in the electronic questionnaire either by the interviewer or the interviewee themselves. In step 132, the question and response and voice recording are stored in the database storage for subsequent auditing.

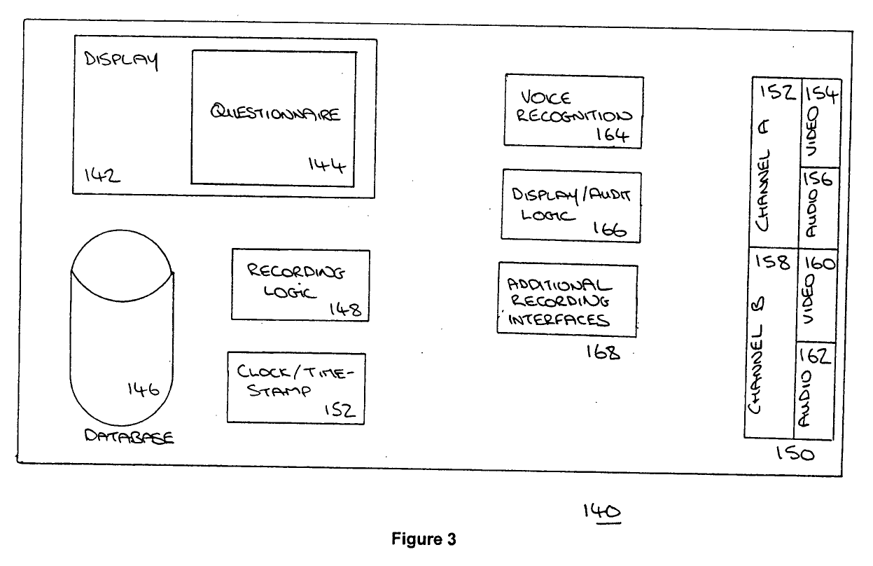

FIG. 3 shows an illustration of a system for audio and video recording and auditing of case histories and interviews, in accordance with an alternate embodiment of the present invention. As shown in FIG. 3, in accordance with an embodiment, the system 140 again comprises a display 142, for displaying an electronic version of the questionnaire 124. The system also comprises a recording interface 150. In accordance with this embodiment, the recording interface includes a plurality of channels, including a first channel 152 (occasionally referred to herein as Channel A or Left Channel), and a second channel 158 (occasionally referred to herein as Channel B or Right Channel). By including two or more channels, with separate microphones associated with each channel, the system can easily differentiate between the interviewers questions and the interviewees responses, for even greater precision. Each recording interface channel can include separate audio 156, 162, or video 154, 160 interface components. A recording logic 148 controls the entire process, and particularly controls the process of storing questions, answers, and/or backing data or images within the system in a database storage 146. In accordance with an embodiment, a clock and time stamping feature 152 is provided to timestamp the answers, for use in subsequent playback and auditing. Optionally, a voice recognition (VR) component 164 is included to allow the system to recognize the answers and ensure that a recorded verbal answer matches that entered by the interviewer into the electronic questionnaire. Optionally also, a display/audit logic 166 is provided to control a visual feedback to the interviewer, in conjunction with the voice recognition component, as described in further detail below, to assist the interviewer in recognizing answers that may have been misunderstood or otherwise recorded incorrectly. Additional recording interfaces 168 may also be provided to receive data from devices in addition to video cameras and microphones, for example from touchpads, data recorders, or other input devices and sensors.

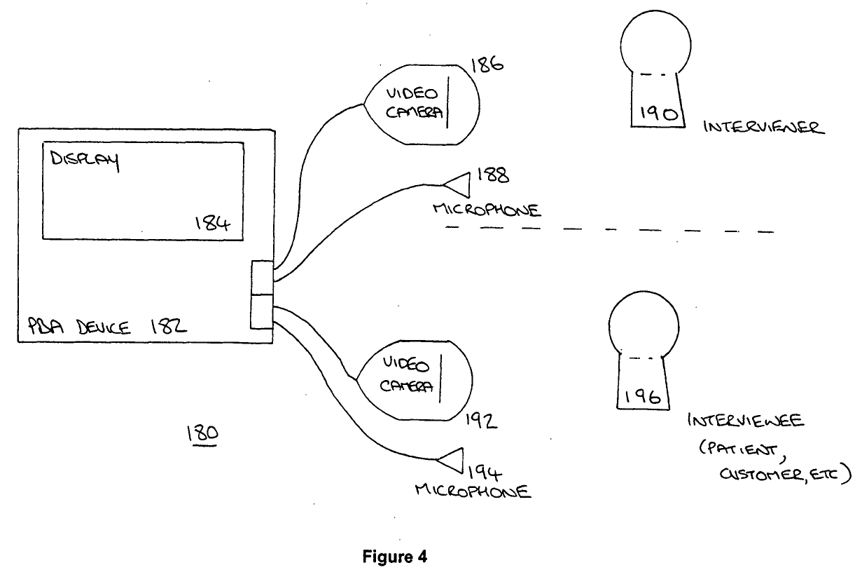

FIG. 4 illustrates an example of how the system may be used in a medical or other interview setting, in accordance with an embodiment of the present invention. As shown in FIG. 4, the system 180 may be provided as a personal digital assistant device (PDA), or similar portable device. In accordance with this embodiment the system may include the digital display 184 within or as part of the device itself. Similarly, the recording interfaces, database storage, recording logic, and other components may be built-in to the device to create a completely portable system. As shown in FIG. 4, the system may be connected to a plurality of video cameras 186, 188 and/or microphone devices 188, 192 for use in recording the questions and answers of the interviewer 190 and interviewee respectively.

Self-Assembling Database File

In accordance with an embodiment, the system can make use of a self-assembling database file structure to store the questionnaire, questions, and/or the interviewees answers. An example of the self-assembling database file structure and method for creating same is disclosed in copending provisional patent application “SYSTEM AND METHOD FOR CREATING AND USING SELF-ASSEMBLING DATABASES”, Inventor Allan Gardiner, application Ser. No. 60/581,250, filed Jun. 18, 2004, incorporated herein fully by reference. In accordance with an embodiment, the self-assembling database file contained a compressed copy of the interview covered by the questionnaire. Very high compressions can be obtained using techniques such as MP3, Real Audio, Microsoft Media format, and other algorithms. The audio and/or video files can be embedded with the questionnaire image and any tabular data. The file type may be created using any of the existing file types that can hold data in addition to the raw image, such as the png file format described in the copending provisional patent application “SYSTEM AND METHOD FOR CREATING AND USING SELF-ASSEMBLING DATABASES”. Other proprietary or non-proprietary file types may also be used, or created with formats that include blocks for tabular or tagged data, image data, and audio or video data.

The stored files can be audited completely as recorded (i.e. the entire 100% of the interview can be audited), or alternatively time markers can be inserted each time a question is answered. These time marks are used during the audit process to replay that portion of the interview that begins several seconds before the question was answered (e.g. 10 seconds), and continue through the answer, to allow the auditor to hear the question, and hear the answer.

The time marker can be tagged data or coded into the audio/video data. The purpose of the time stamping of each question reduces the time to audit the question. For example, a 15 question questionnaire may take 10 minutes to complete. An un-stamped audit would obviously take 10 minutes to complete. However, when using time stamps with a 10 second interval , the audit will take approximately a quarter of the time. Additionally, the system can be adapted to permit hearing or seeing the interviewer/interviewee interaction by clicking on an individual question being audited.

A single file created by these methods is superior to multiple files linked by tags such as XML or hypertext. The single file can be used for audit and data base reconstruction purposes. Auditing separate files can be done using the single file as the original file, for comparison. Multiple formats can be included in the file to accommodate compressed data either lossless or with accepted losses associated with the compression algorithm, (e.g., MP.3) from many sources, replay and review/audit functions are handles by software written to utilize the tags, times stamps, and the navigating codes.

While self assembling database capabilities are maintained through the use of single files that do not require outside reference whenever possible, outside references may be required for large data files which may be too large for complete reconstruction. Multiple smaller, but independent files may be reassembled back into larger files by means of the time stamp, etc.

Implementations and Variations

The above description defines basic embodiments needed to practice the invention. Modifications to the above include the following:

Use of Two or More Sound Channels

In accordance with this embodiment, one channel is used for the interviewer, and an alternate second channel is used for the interviewee, using personal microphones, video cameras or other input devices. Using two or more channels simplifies the identification of which person is talking at any particular moment in time. Additionally, the subject and interviewer do not have to be near each other. As described above, automated voice recognition software can also be used to perform an electronic analysis of the questions and answers. A training period can be performed beforehand to improve the accuracy of the recognition process. Time stamping can speed transaction recovery by mixing voice recognition to refine the time stamp accuracy. For example, in practical use the audio answer from the interviewee may be communicated 3 to 5 seconds before the actual entry of the data on the electronic form. Voice recognition can be used to refine the output window such that the time window ends after the audio answer is received on the interviewee channel.

Use of a Start/Stop Function

In accordance with this embodiment, the interviewer is provided with a start/stop function to isolate the question/answer at the time of recording. This method prevents unwanted conversations from being included in the record. However, it will be evident that some applications may be better documented by recording the entire interview, and not just portions thereof. The value of the time stamped answers is high for a long continuously recorded session.

Comparison of Interviewee Answers Using Voice Recognition

In accordance with this embodiment, an interviewee's answer can be compared by using voice recognition, and then displaying or comparing the interviewers entry with the recognized answer on the interviewees channel. Visual confirmation of the interview is used to catch a high percentage of incorrectly entered data. For example, the answer box can be shaded green if the voice recognition matches the entered answer, or alternatively can be shaded red if there is a difference. This method of confirmation assures the interviewer in real time that the information being entered is correct.

Time-Window Revision of Complex Answers

In accordance with this embodiment, answers that are neither a simple “yes” or “no” can be reviewed using the time window approach or by voice recognition print out. The audio track is also preserved for human intervention if the answer is unrecognizable by the system.

Self-Administered Questionnaire

In accordance with this embodiment, the interviewee is provided with a self-administered questionnaire. The questionnaire is displayed as described above on the display, but the system acts as the interviewer. The self-administered questionnaire can be programmed to capture the audio and/or video track and preserve it for auditing. For example, in a typical session the interviewee may be asked to say “yes”, “no”, or some other word in response to a question. The voice recognition software answers the question for the interviewee in text format, or for comparing with the entered answer. The interviewee can confirm his/her answer by pressing a button or by a voice acknowledgment. This type of voice recognition challenge/answer/confirm is similar to that used in voice-assisted telephone systems.

Form Revision

In accordance with this embodiment, form revision is a feature used by the system to require a statement by the person reviewing the answers. Changing answers later in the interview can provide historical record of changes by means of time stamps. The statement may be the old or new answer identified, or an explanation of why the question is being revised. The review of revised forms can be simplified by allowing the voice recognition component to refine the playback window. Any complex or additional reasoning for a revision can be included in the stored file.

Single File Storage

In accordance with this embodiment, a single file is used for the storage of data. Advantages to having a single file include that the integrity of the tagged images and the sound can be assured by means of techniques such as encryption of the data, watermarks, digital signatures, or other protection methods. Additionally, multiple files can be concatenated using the automatic assembling databases described herein and in U.S. application Ser. No: 60/581,250, herein incorporated fully by reference.

Use of Third-Party Video and Other Video Environments

In accordance with this embodiment, third-party video is used as the data source. Medical clinical trials are designed to prevent interviewee identification which makes audio methods more attractive than video methods. Questionnaires and data collection in less protective environments may favor video auditing. For example, questioning of suspects at a police station can use the video time stamped to the questions method. The video can be taken from the recording system which is already in place at the police station for this type of questioning, without the need to add a second video system.

Additional Testing Interfaces and Data Types

In accordance with this embodiment, additional testing interfaces are provided. Alternatively, an audio or video recording of a particular patient attribute is used. Video capture for patient testing can be automatically made part of the single stored file. For example, neurologically challenged people perform tests such as finger tapping that identify how many times they can open and close two fingers within a 10 second period. The video capture of the test associated with the question can make counting of taps much easier by playing back a defined window of time (for example, 10 seconds). The capture form can be interactive for subsequent revision or entry of observers data or recorded statement after the interviewee has departed. Voice recognition can be added to simplify the entry of text information resulting from voice addition. For example, the interviewer may count the taps and enter the number on the form after the interviewee has gone. Alternatively, automated image recognition programs can be used to count fingertaps. In some embodiments, fingertaps can be measured using an accelerometer placed on a finger. Results from such programs can be added to the database. The simple time based tests can be self-administered at a workstation using instruction presented by multimedia. The measurement on a new submit can be done by a human interviewer on the first visit and a later visit and the self-administered version on in-between visits. This permits collection of data that would be too expensive to otherwise collect.

Video Clip Association

In accordance with this embodiment, a video clip or multiple clips may be associated with the questionnaire, or the answers thereto, and recorded or embedded with the file. These methods of collecting, reviewing, revising, auditing video or audio data into interactive single files permits automatic self assembly of database as previous described. Additional features of this idea extend to doing before/after comparisons. For example, mobility tests such as gait toward or away, or heel-to-toe can be automatically displayed concurrently in a play back mode. Each video clip is associated with its tags and question or instruction for retrieval.

Collection of Test or Sensor Data

In accordance with this embodiment, collection of test data such as from an electro cardiogram or Doppler bloodflow meter, accelerometer or other measurement device can be included in the file for later retrieval or comparison. This function is also a part of the self assembly database, described in further detail in copending provisional patent application “SYSTEM AND METHOD FOR CREATING AND USING SELF-ASSEMBLING DATABASES”, Inventor Allan Gardiner, application Ser. No. 60/581,250, filed Jun. 18, 2004, incorporated herein fully by reference.

Multiple Data Streams with Single File Capture

In accordance with this embodiment, the methods of captures and file building presented here can be applied to any data collection system where multiple data streams are available at the time of capture. Construction of a single file can be a function in addition to separate files generated and stored separately.

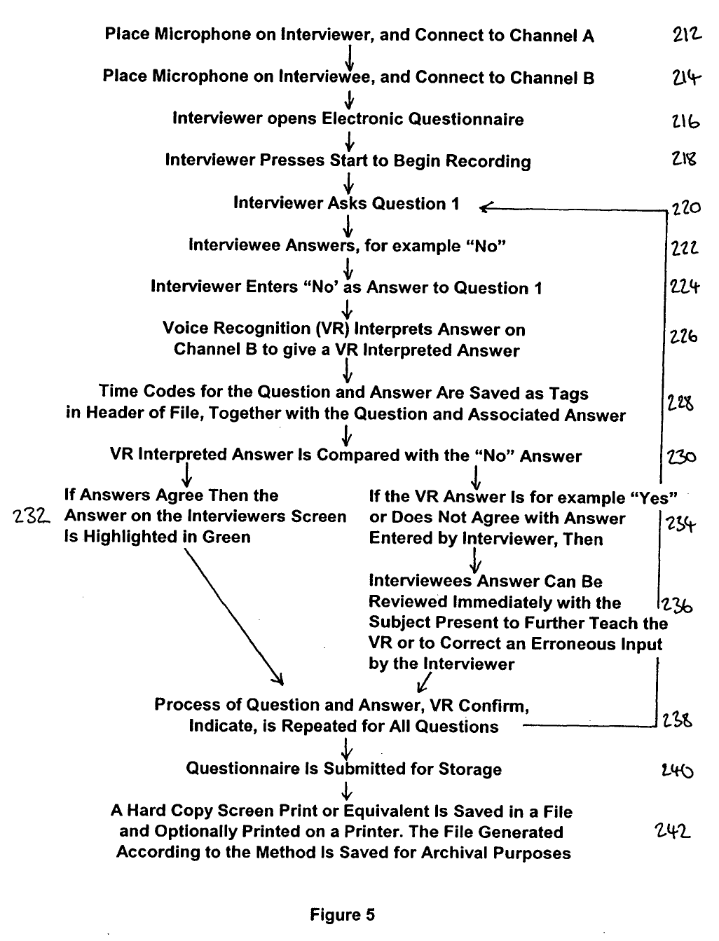

FIG. 5 shows a flowchart of a method of capturing audio video during a multiple question interview, together with voice recognition, in accordance with an embodiment of the present invention. As shown in FIG. 5, in this example the first step 212 is to place a microphone on or directed toward the interviewer connected to a first channel or Channel A, and in step 214 to place a microphone on the interviewee connected to a second channel or Channel B. In step 216, one of the parties (typically the interviewer, although in a self-conducted interviewer the system itself may take the role of interviewer), opens the electronic questionnaire. In practical situations, an interviewer may, as a preliminary step, teach the interviewee how to answer a question in a manner that the voice recognition (VR) system used can interpret the response correctly. In step 218, the interviewer then presses a start button or function to begin recording. In step 220, the interviewer asks a first question, Question 1. If, for example, the interviewee in step 222 answers “No”, then, in step 224 the interviewer enters “No” in the electronic questionnaire as the answer to Question 1. In accordance with an embodiment, in step 226 the VR interprets the answer on channel B to give a VR interpreted answer. Optionally, in step 228 time codes for the question and answer are saved as tags in the header of the file along with the question and associated answer. In step 230, the VR interpreted answer is compared with the answer given by the interviewee and recorded by the interviewer (in this example the “No” answer). In step 232, if the two answers agree, then the answer on the interviewer's screen is highlighted in green. It can be appreciated that the interviewee's screen can also display the VR-confirmed answer. This can be performed by the display/audit logic described above. In step 234 if the two answers differ, for example if the VR answer is “Yes” and does not agree with the “No” entered by the interviewer then in step 236 the interviewee answer can be reviewed immediately with the interviewee present to further teach the VR, or to correct an erroneous input by the interviewer. In step 238, the process of question and answer, VR confirmation, and display indication is repeated for all questions. In step 240, the questionnaire is submitted for storage, and optionally in step 242, a hard copy screen print or equivalent is saved in a file or printed to a printer.

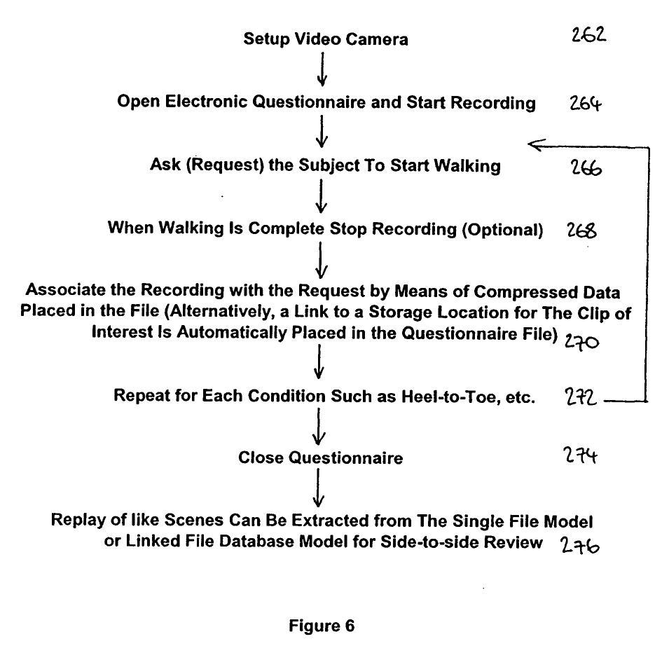

FIG. 6 shows a flowchart of a method of recording and displaying gait change progress in an interviewee, in accordance with an embodiment of the present invention. This flowchart illustrates how the invention may be used to record case history information beyond mere words and verbal answers to questions. In some instances, a video recording, accelerometer or position tracking recordings can augment an interviewee's answers in a way that cannot be expressed in words. In some embodiments, several accelerometers or other devices can be placed on feet, legs, lower body, shoulders and/or head. As shown in FIG. 6, in step 262 the video camera, or some other type of recording device, is set up. In step 264, the interviewer opens the electronic questionnaire and starts recording. The process then continues in a manner similar to above, with a series of questions, instructions answers and/or actions. For example, in step 266, the interviewer may ask the interviewee to start walking. In step 268, when the action (walking) is complete, the interviewer can stop recording. In step 270, the system associates the recording with the request by means of compressed data placed in the file. Alternatively, a link to a storage location for the clip of interest can be automatically placed in the questionnaire file. In step 272, the process is repeated for each relevant condition (such as heel-to-toe, etc.). In step 274, the questionnaire is then closed. In step 276, a replay of like scenes can be subsequently extracted from the single file model or linked file database model for side-to-side review. It can be appreciated that these types of embodiments can be applied to any process, such as range of motion, tremor, or industrial processes.

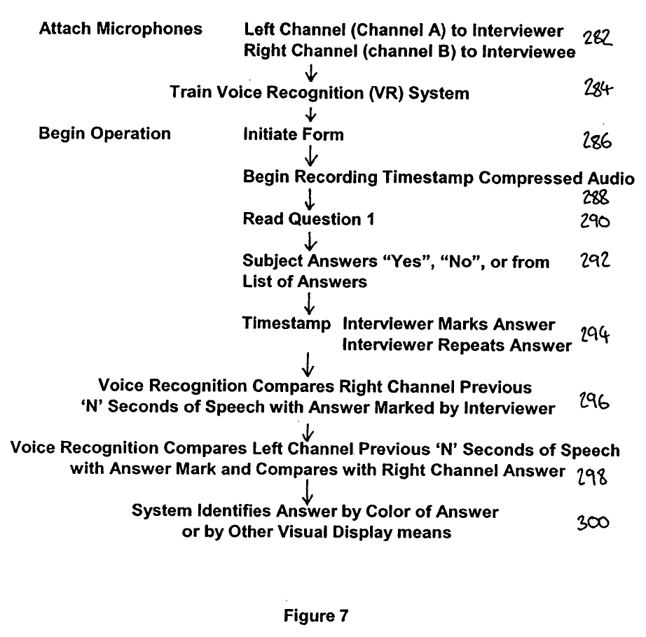

FIG. 7 shows a flowchart of a method of using voice recognition to audit answers, in accordance with an embodiment of the present invention. As shown in FIG. 7, in step 282, the interviewer attaches the microphone (or microphones), for example the left channel can be connected to the interviewer, and the right channel to the interviewee. In step 284, the voice recognition (VR) component can be trained to recognize the interviewees responses. The actual interview operation then begins. In step 286, a questionnaire form is initialized by displaying it on a screen. In step 288, the recording is started. In step 290, the interviewer reads the first question, (Question 1). In step 292 the interviewee answers any of “Yes”, “No”, or from a list of other alternative answers. In step 294 the interviewer marks the answer, which is time stamped, and then repeats it. In step 296, the Voice Recognition component compares the previous N seconds of speech as recorded on the right channel with the answer marked by interviewer. In step 298, the system then compares the Left Channel previous n seconds of speech with the answer, and compares that with the Right Channel answer. In step 300, the system identifies the answer by color of answer or by another form of visual display. These methods can increase the likelihood that correct answers are marked and that the original (e.g., audio) data may be audited.

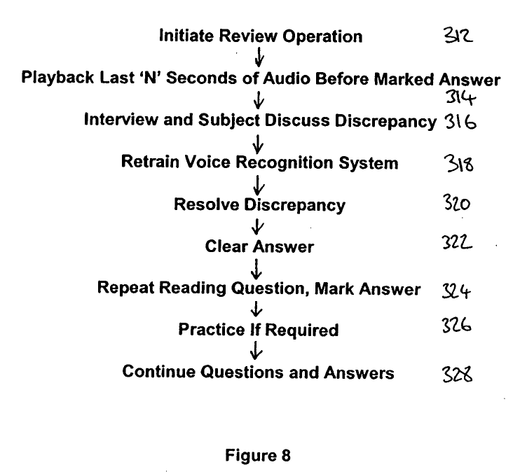

FIG. 8 shows a flowchart of a method of conducting a review operation, in accordance with an embodiment of the present invention. In step 312, the review operation is initiated. In step 313, an image of the form questions and answers can be presented on the screen with software that allows the reviewer to rapidly select desired questions or events for replay. In step 314 the system plays back the last N seconds of audio before the marked answer. In step 316, the interviewer and the interviewee discuss any discrepancy. If the discrepancy is believed to be a result of the VR inaccuracy, then in step 318 the voice recognition component is retrained. In step 320 the discrepancy is resolved, and in step 322 the answer is cleared. In step 324 the interviewer repeats reading the question, and marking the answer. In step 326, the parties may practice if required. In step 328, the process continues with further questions and answers.

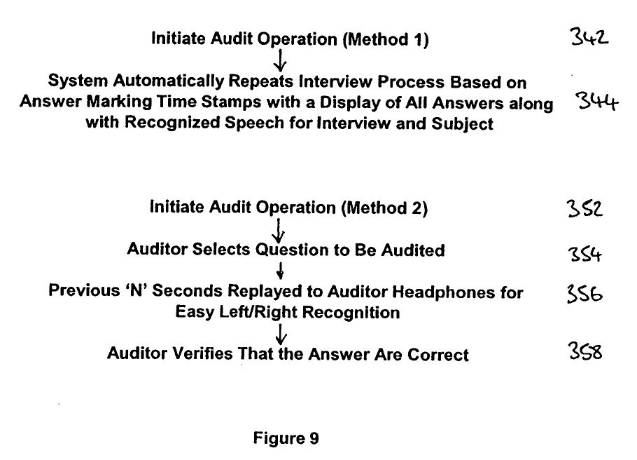

FIG. 9 shows a flowchart of a method of conducting an audit operation, in accordance with an embodiment of the present invention. FIG. 9 illustrates two alternative methods. As shown in FIG. 9, in the first method, Method 1, in step 342 the audit operation is initiated. In step 344 the system automatically repeats the process based on Answer Marking time stamps, with a display of all answers together with recognized speech for the interviewer and interviewee. Also as shown in FIG. 9, in the second method, Method 2, in step 352 the audit operation is initiated. In step 354 the auditor selects a question to be audited. In step 356, the previous N seconds are replayed to the auditor headphones for easy left/right recognition. In step 358 the auditor verifies that the answers are correct. In this manner random or complete verification may be easily accomplished.

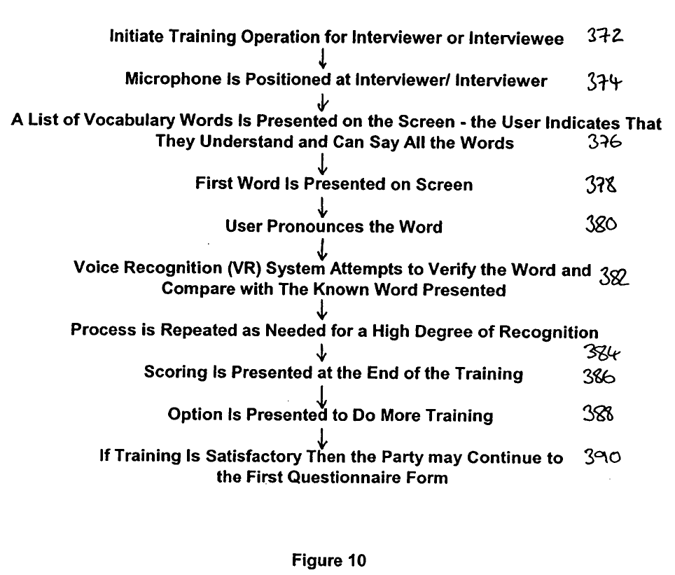

FIG. 10 shows a flowchart of a method of training the voice recognition system, in accordance with an embodiment of the present invention. As shown in FIG. 10, in step 372 the process of Training for a user (Interviewee or Interviewer) is initiated. In step 374, a microphone is positioned at the user. In step 376, a list of Vocabulary Words is presented on the screen. In step 378, the first word is presented to the user. In step 380, the user says the word. In step 382, the voice recognition system attempts to verify the word and compare it with the known word presented. In step 384 these steps are repeated as needed for a high degree of recognition accuracy. In step 386, at the end of the training process a scoring is presented. In step 388, the option is then presented to do more training, or in step 390 if the training is satisfactory then the user can go on to the first form or set of questions.

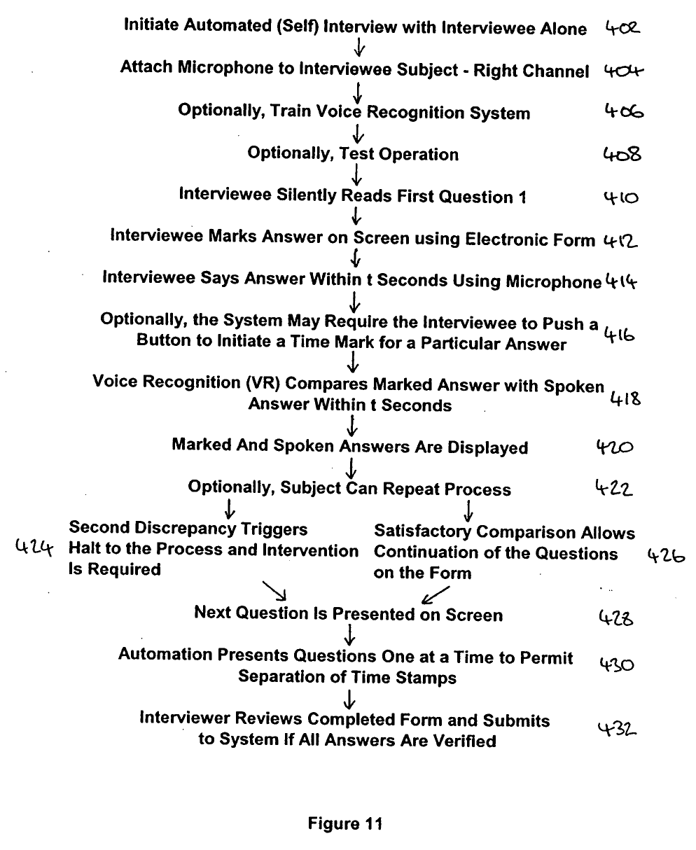

FIG. 11 shows a flowchart of a method of allowing an interviewee to conduct a self-interview or self-questionnaire, in accordance with an embodiment of the present invention. As shown in FIG. 11, in step 402 the process of performing an Automated Interview with Interviewee Alone is initiated. In step 404 a Microphone is attached to or directed towards the Interviewee (in accordance with an embodiment to the Right Channel). In step 406 the voice Recognition System is optionally trained, and in step 408 optionally tested. In step 410 the Interviewee Reads silently to themselves the first question (Question 1). In step 412 the Interviewee marks their answer on the screen within the electronic form. In step 414 the interviewee then says the answer aloud within t seconds using microphone. Optionally, in step 416, the system may require the interviewee to push a button to initiate at time mark for a particular answer. In step 418 the Voice recognition system compares the marked answer with the spoken answer within t seconds. In step 420 the marked and spoken answers are displayed. In step 422, the interviewee can choose to repeat the process. In step 424, any second discrepancy triggers a halt to the process and intervention is required. However, in step 426, a satisfactory comparison allows the process to continue. In step 428 the Next question is presented on the screen. In step 430 automation presents questions one at a time to permit separation of time stamps. In step 432 the Interviewer reviews completed form and submits it to system if all answers are verified.

In other embodiments, one can record events that are not questions and answers. For example, time and motion studies typically utilize an observer who marks events of several types (e.g., start, step 1, step 2, step 3, good part, bad part, stop. Each event can be time stamped to audit by video, audio, or other data input. Events can be placed in tags according to methods described in U.S. application Ser. No.: 60/581,250 (incorporated herein fully by reference), and placed in a database for easy computation and preservation of data, determination of event times and elapsed times can be done using automated recognition of video, audio or other data.

The present invention may be conveniently implemented using a conventional general purpose or a specialized digital computer or microprocessor programmed according to the teachings of the present disclosure. Appropriate software coding can readily be prepared by skilled programmers based on the teachings of the present disclosure, as will be apparent to those skilled in the software art.

In some embodiments, the present invention includes a storage medium (media) having medical information stored thereon/in which can be used by program a computer to perform processes of the present invention. The storage medium can include, but is not limited to, any type of disk including floppy disks, optical discs, DVD, CD-ROMs, microdrive, and magneto-optical disks, ROMs, RAMs, EPROMs, EEPROMs, DRAMs, VRAMs, flash memory devices, magnetic or optical cards, nanosystems (including molecular memory ICs), or any type of media or device suitable for storing instructions and/or data.

The foregoing description of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to explain the principles of the invention and its practical application, thereby enabling others skilled in the art to understand the invention for various embodiments and with various modifications that are suited to the particular use contemplated.

### Industrial Applicability

The invention provides techniques for recording interviews and other medical information in a medical, clinical trial, or other commercial setting. Such techniques find applicability in the medical information and medical research industries. The system allows for quick interviewing of a patient or customer, by a single interviewer, yet ensures that the interviewees answers are reliably recorded in real-time, and with the confidence that a backing audio and/or video recording is kept for auditing purposes. This invention also provides systems and methods for creating databases containing medical information via video, audio and other measurement or monitoring methods.

I claim:

1. A system for auditing of medical information of a subject, comprising:
a recording interface that records subject's medical information associated with a time stamp in a single file;
a database that saves said medical information;
an electronic auditor adapted to coordinate time stamps; and
a replay device to present said information based upon time stamps.

2. The system of claim 1, wherein said information includes questions and answers.

3. The system of claim 1, wherein said information includes video and/or audio information.

4. The system of claim 1, wherein said information includes one or more measured variables.

5. The system of claim 1 wherein said information is stored as a self-assembling database file in the database storage.

6. The system of claim 1 wherein the entire system is contained within a portable device.

7. The system of claim 2 wherein the question comprises an electronic image, and wherein a copy of the subject's answer is recorded with or embedded into that electronic image prior to storage within the database storage device.

8. A method for auditing of medical information of a subject, comprising the steps of:
displaying on a display an electronic version of a questionnaire containing questions to be given to or asked of a subject;
recording via a recording interface, the subject's answer to a question, said answer associated with a time stamp;
saving in a database storage a copy of the question together with a copy of the answer given by the subject to the question and said time stamp; and
replaying said information based upon said time stamps.

9. The method of claim 8 wherein the recording interface is an audio recording interface that saves an audio copy of the subject's answer to a question in the database storage device.

10. The method of claim 8 wherein the recording interface is a video recording interface that saves to the database storage a video copy of the subject's answer.

11. The method of claim 8 wherein the questions and answers are stored in the database storage as a self-assembling database file.

12. The method of claim 8 wherein the entire system is contained within a portable device.

13. The method of claim 8 wherein the questionnaire includes an electronic image, and wherein a copy of the subject's answer is recorded with or embedded into that electronic image prior to storage within the database storage.

14. The system of claim 1, wherein said information comprises revised information having all time stamps associated with said revised information and of said original information associated with said revised information.

15. A system for audio and video auditing of medical information of a subject, comprising:
a portable device, said portable device comprising:
a display that displays an electronic version of a questionnaire containing questions to be given to or asked of a subject, wherein the questionnaire includes an electronic image;
a recording interface that records the subject's answer to a question;
a database storage that saves a copy of the question as a self-assembling database together with a copy of the answer given by the subject to the question, wherein a copy of the subject's answer is recorded with or embedded into that electronic image prior to storage within the database storage;
a clock and a time stamp feature that stamps the subject's answer with a time stamp for subsequent auditing; and
a microphone for communicating a subject's answer to the recording interface.

16. The system of claim 1, wherein said medical information comprises a plurality of questions and answers.

17. The method of claim 8, wherein said medical information comprises a plurality of questions and answers.

18. The system of claim 15, wherein said medical information comprises a plurality of questions and answers.

19. The system of claim 1, wherein said information further comprises data obtained from one or more measurement devices.

20. The method of claim 8, wherein said medical information additionally comprises data obtained from one or more measurement devices.

21. The system of claim 15, wherein said medical information further comprises data obtained from one or more measurement devices.

22. The system of claim 1, wherein said database further comprises information from an interviewer.

23. The method of claim 8, wherein said database further comprises information from an interviewer.

24. The system of claim 15, wherein said database further comprises information from an interviewer.
